(19) United States
(12) Patent Application Publication
(10) Pub. No.: US 2026/0102573 A1
(43) Pub. Date: Apr. 16, 2026

(54) HEMOSTATIC POWDER DELIVERY DEVICES AND METHODS OF USE

(71) Applicant: BOSTON SCIENTIFIC MEDICAL DEVICE LIMITED, Galway (IE)

(72) Inventors: Rajivkumar SINGH, Pune (IN); Sharath Kumar G, Kanakapura (IN); Nachiket GOLE, Pune (IN); Deepak Kumar SHARMA, Muzaffarnagar (IN)

(73) Assignee: BOSTON SCIENTIFIC MEDICAL DEVICE LIMITED, Galway (IE)

(21) Appl. No.: 19/357,382

(22) Filed: Oct. 14, 2025

Related U.S. Application Data
(60) Provisional application No. 63/707,926, filed on Oct. 16, 2024.

(51) Int. Cl. A61M 11/02; A61M 39/22; A61M 39/00

(52) U.S. Cl. CPC A61M 11/02; A61M 39/227; A61M 2039/0009; A61M 2202/0007; A61M 2202/064; A61M 2205/3331

(57) ABSTRACT

A device for delivering an agent includes a valve assembly including an enclosure for storing the agent, an agitation mechanism rotatably disposed inside the enclosure, and a tube disposed inside the enclosure and extending through the agitation mechanism. The tube includes a channel having a longitudinal length defined between a first opening at a first end of the tube and a second opening at a second end of the tube. The agitation mechanism is configured to direct a fluid received in the enclosure towards the agent stored therein in response to rotating about the tube, thereby agitating the agent within the enclosure. The tube is configured to receive a mixture of the fluid and the agent at the second opening and direct the mixture through the channel for delivery out of the enclosure via the first opening.

### CROSS-REFERENCE TO RELATED APPLICATIONS

This application claims the benefit of priority to U.S. Provisional Application No. 63/707,926, filed on October 16, 2024, which is incorporated by reference herein in its entirety.

### TECHNICAL FIELD

Various embodiments of the present disclosure relate generally to devices and methods for delivering agents. More specifically, this disclosure relates to devices for delivery of powdered agents, such as hemostatic agents.

### BACKGROUND

In certain medical procedures, it may be necessary to minimize or stop bleeding internal to the body. For example, an endoscopic medical procedure may require hemostasis of bleeding tissue within the gastrointestinal tract, for example in the esophagus, stomach, or intestines. During an endoscopic procedure, a user inserts a sheath of an endoscope into a body lumen of a patient. The user utilizes a handle of the endoscope to control the endoscope during the procedure. Tools may be passed through a working channel of the endoscope via, for example, a port in the handle, to deliver treatment at the procedure site near a distal end of the endoscope. The procedure site is remote from the user.

To achieve hemostasis at the remote site, a hemostatic agent may be delivered by a delivery device inserted into the working channel of the endoscope. Agent delivery may be achieved, for example, through mechanical systems. Such systems, however, may require numerous steps or actuations to achieve delivery, may not achieve a desired rate of agent delivery or a desired dosage of agent, may result in the agent clogging portions of the delivery device, may result in inconsistent dosing of the agent, and/or may not result in the agent reaching the treatment site deep within the gastrointestinal tract. The current disclosure may solve one or more of these issues or other issues in the art.

### SUMMARY OF THE DISCLOSURE

Aspects of the disclosure relate to, among other things, systems, devices, and methods for a medical device including a valve assembly for delivering agents. More specifically, this disclosure relates to devices for delivery of powdered agents, such as hemostatic agents, via a valve assembly that includes a cavity for storing the hemostatic agent and an agitation mechanism that is movably disposed inside the cavity to facilitate delivery of the hemostatic agent out of the device. Each of the aspects disclosed herein may include one or more of the features described in connection with any of the other disclosed aspects.

According to an example, a device for delivering an agent includes a valve assembly including an enclosure for storing the agent, an agitation mechanism rotatably disposed inside the enclosure, and a tube disposed inside the enclosure and extending through the agitation mechanism; wherein the tube includes a channel having a longitudinal length defined between a first opening at a first end of the tube and a second opening at a second end of the tube; wherein the agitation mechanism is configured to direct a fluid received in the enclosure towards the agent stored therein in response to rotating about the tube, thereby agitating the agent within the enclosure; wherein the tube is configured to receive a mixture of the fluid and the agent at the second opening and direct the mixture through the channel for delivery out of the enclosure via the first opening.

Any of the devices described herein may include any of the following features. The agitation mechanism includes a propeller including a main body; and a plurality of blades extending radially outwards from the main body. The main body includes a top end including a planar surface covering the plurality of blades and an opening extending through the planar surface, the opening being configured to surround the first end of the tube and a bottom end that is open to expose the plurality of blades, the bottom end including a seat disposed adjacent to the bottom end along the plurality of blades. The main body includes a top end that is open to expose the plurality of blades, the top end including an opening configured to surround the first end of the tube, and a bottom end including a planar surface covering the plurality of blades, wherein the opening extends through the planar surface, wherein the agitation mechanism includes a base and a plurality of arms extending between the main body to the base, the plurality of arms being configured agitate the agent within the enclosure in response to the agitation mechanism rotating about the tube. The base is offset from the second end of the tube to form a gap, wherein the base is configured to direct the mixture into the channel for delivery from the tube via the gap. The base includes an agitator extending outwardly from the base towards the second end of the tube, the agitator being configured agitate the agent within the enclosure in response to the agitation mechanism rotating about the tube. Each of the plurality of arms includes a helical configuration extending between the main body and the base. The main body includes a top end including a top planar surface covering the plurality of blades and an opening extending through the top planar surface, the opening being configured to surround the first end of the tube, and a bottom end including a bottom planar surface covering the plurality of blades such that the plurality of blades are disposed between the top planar surface and the bottom planar surface, wherein the opening extends through the bottom planar surface. The main body includes at least one slot formed between a pair of adjacent blades of the plurality of blades, wherein the at least one slot is configured to direct the fluid received within the main body outwards from between the top planar surface and the bottom planar surface towards the agent. The main body includes at least one vent formed within the at least one slot between each of the pairs of adjacent blades, wherein the at least one vent is configured to direct the fluid received within the at least one slot downwards through the bottom planar surface towards the agent. The tube includes a platform between the first end and the second end, the platform is configured to receive the agitation mechanism on the tube. The enclosure includes a first fastening mechanism, and the tube includes a second fastening mechanism at the first end that is configured to removably couple the tube to the enclosure, thereby suspending the second end of the tube inside the enclosure. The enclosure includes an upper body and a lower body that are removably coupled to one another to form a cavity, the tube and the agitation mechanism are disposed inside the cavity. The tube extends towards a bottom surface of the enclosure to create a gap between the second end of the tube and the bottom surface, the tube is configured to receive the mixture within the channel via the gap. A connector assembly is removably coupled to the valve assembly, wherein the connector assembly includes a port configured to receive an instrument to clear an outlet of the device of residual agent.

According to another example, a device for delivering an agent includes a valve assembly including an upper body, a lower body removably coupled to the upper body to define an enclosure, and a tube disposed inside the enclosure and extending between the upper body and the lower body; wherein the tube includes a first end mounted to the upper body and a second end opposite to the first end that is suspended inside the lower body to form a gap; wherein the upper body is configured to guide a pressurized fluid received in the valve assembly towards the lower body in a cyclonic motion, and the lower body is configured to guide the pressurized fluid received from the upper body in the cyclonic motion towards the agent, thereby agitating the agent with the pressurized fluid; wherein the tube is configured to receive a mixture of the pressurized fluid and the agent at the second end via the gap for delivery from the lower body to the upper body for exiting the enclosure.

Any of the devices described herein may include any of the following features. The gap includes an offset distance formed between a bottom surface of the lower body and the second end of the tube. The upper body includes an interior surface that is configured to direct the pressurized fluid received within the upper body downwards towards the lower body in the cyclonic motion.

According to a further example, a device for delivering an agent includes a valve assembly including an enclosure, a tube disposed inside the enclosure, and an agitation mechanism rotatably mounted to the tube, the enclosure is defined by an upper body that is configured to receive a pressurized fluid and a lower body that is configured to store the agent; wherein the agitation mechanism is configured to rotate about the tube in response to the upper body receiving the pressurized fluid to propel the pressurized fluid downwards towards the agent stored in the lower body; wherein the tube includes a channel that is in fluid communication with the lower body, such that the tube is configured to receive a mixture of the pressurized fluid and the agent within the channel for delivery out of the enclosure.

Any of the devices described herein may include any of the following features. The agitation mechanism includes a propeller having a main body and a plurality of blades extending radially outwards from the main body, each of the plurality of blades including an interior surface that is configured to capture the pressurized fluid, thereby causing the main body to rotate around the tube.

It may be understood that both the foregoing general description and the following detailed description are exemplary and explanatory only and are not restrictive of the invention, as claimed. As used herein, the terms “comprises,” “comprising,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “diameter” may refer to a width where an element is not circular. The term “top” refers to a direction or side of a device relative to its orientation during use, and the term “bottom” refers to a direction or side of a device relative to its orientation during use that is opposite of the “top.” The term “exemplary” is used in the sense of “example,” rather than “ideal.” The term “approximately,” or like terms (e.g., “substantially”), includes values +/- 10% of a stated value.

### BRIEF DESCRIPTION OF THE DRAWINGS

The accompanying drawings, which are incorporated in and constitute a part of this specification, illustrate aspects of this disclosure and together with the description, serve to explain the principles of the disclosure.

FIG. 1 shows an exemplary delivery device, according to one or more embodiments.

FIG. 2 shows an exploded view of an exemplary valve assembly of the delivery device of FIG. 1, according to one or more embodiments.

FIG. 3 shows a partial top view of the valve assembly of FIG. 2, according to one or more embodiments.

FIG. 4 shows a partial side view of the valve assembly of FIG. 2, according to one or more embodiments.

FIG. 5 shows an exploded partial view of an agitation mechanism and an outlet tube of the valve assembly of FIG. 2, according to one or more embodiments.

FIG. 6 shows a cross-sectional view of the agitation mechanism and outlet tube of the valve assembly of FIG. 2 taken along line 6-6 of FIG. 4, according to one or more embodiments.

FIG. 7 shows a perspective view of the valve assembly of FIG. 2 and an exemplary connector assembly, according to one or more embodiments.

FIG. 8 shows a perspective view of another exemplary agitation mechanism of the valve assembly of FIG. 2, according to one or more embodiments.

FIG. 9 shows a perspective view of another exemplary agitation mechanism of the valve assembly of FIG. 2, according to one or more embodiments.

FIG. 10 shows a perspective view of another exemplary agitation mechanism of the valve assembly of FIG. 2, according to one or more embodiments.

FIG. 11 shows a perspective view of another exemplary agitation mechanism of the valve assembly of FIG. 2, according to one or more embodiments.

FIG. 12 shows a perspective view of another exemplary agitation mechanism of the valve assembly of FIG. 2, according to one or more embodiments.

FIG. 13 shows a bottom view of the agitation mechanism of FIG. 12, according to one or more embodiments.

FIG. 14 shows an exploded view of another exemplary valve assembly of the delivery device of FIG. 1, according to one or more embodiments.

FIG. 15 shows a partial top view of the valve assembly of FIG. 14, according to one or more embodiments.

FIG. 16 shows a partial side view of the valve assembly of FIG. 14, according to one or more embodiments.

### DETAILED DESCRIPTION OF EMBODIMENTS

Embodiments of this disclosure relate to dispensing devices having valve assemblies for releasing a material (e.g., a powdered agent) to a site of a medical procedure. In other words, the valve assemblies may be configured and operable to move stagnant material and facilitate proper dispensing of the material. The valve assembly may include one agitation mechanism rotatably disposed inside an enclosure, in order to combine the agent and a fluid. The enclosure may store a predetermined amount of the agent, and may be in fluid communication with a pressurized fluid medium source (e.g., a gas canister). Upon release of a pressurized fluid into the enclosure, the agent and the fluid may be combined due to the movement agitation mechanism within the enclosure. In some embodiments, movement of the agitation mechanism may be caused by the pressurized fluid received within the enclosure.

The agent may be in fluid communication with the pressurized fluid through an inlet conduit and/or opening formed by the valve assembly. Accordingly, when the agent is combined with pressurized fluid, the valve assembly delivers the combination of the agent and the pressurized fluid through an outlet conduit to a target treatment site during a medical procedure. Aspects of the dispensing device and valve assembly, such as the agitation mechanism, may facilitate agitation and combination of the agent and the pressurized fluid prior to dispensing to prevent or minimize clogging of the dispensing device during delivery. The valve assembly may be configured and operable to move the agent that may be stored in a stagnant state and facilitate proper dispensing of the agent out of the dispensing device.

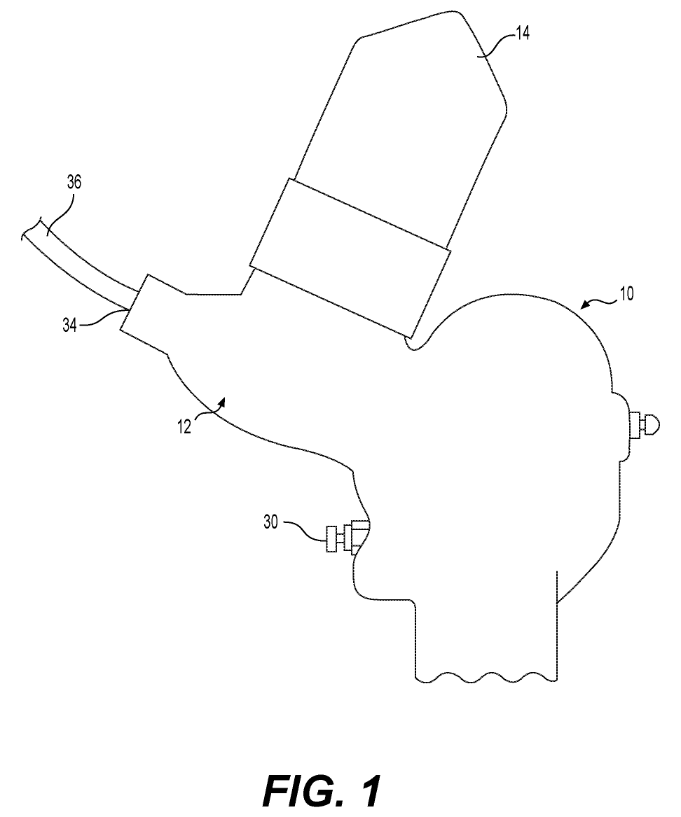

FIG. 1 shows a delivery system 10, which may be a powder delivery system. Delivery system 10 may include a handle body 12. Handle body 12 may include, or may be configured to receive, an enclosure 14 (or other source or container) storing a material (e.g., a powdered agent). Enclosure 14 may be coupled to handle body 12 for providing the agent to handle body 12, or a lid/enclosure of the agent may be screwed onto, or otherwise coupled to, enclosure 14 for supplying the agent to enclosure 14. The agent may be, for example, a powdered agent, such as a hemostatic agent. The agent may alternatively be another type of agent or material, or form of agent (e.g., a liquid or gel agent), and may have any desired function. Enclosure 14 may be removably attached to other components of delivery system 10, including components of handle body 12.

Handle body 12 may have a variety of features, to be discussed in further detail herein. U.S. Patent Application No. 16/589,633, filed October 1, 2019, published as U.S. Patent No. 11,642,281 B1 on May 9, 2023, the disclosure of which is hereby incorporated by reference in its entirety, discloses features of exemplary delivery devices and systems. The features of this disclosure may be combined with any of the features described in the above-referenced patent. The features described herein may be used alone or in combination and are not mutually exclusive. Like reference numbers and/or terminology are used to denote similar structures, when possible.

Still referring to FIG. 1, delivery system 10 may include an actuation mechanism 30 used to activate flow of a pressurized fluid (e.g., gas) from a pressurized medium source (e.g., gas canister) in fluid communication with delivery system 10. Actuation mechanism 30 may be selectively actuated (e.g., manually depressible) or otherwise moved or actuated to control delivery of a material (e.g., a powdered agent) and/or the pressurized fluid. The pressurized fluid alone, or a combination of a powdered agent and the pressurized fluid, may be delivered from an outlet 34 of handle body 12. Outlet 34 may be in fluid communication with a delivery conduit, for example a catheter 36 or another component for delivering the combination of agent and fluid to a desired location within a body lumen of a patient. Delivery system 10 may include a valve assembly for selectively controlling the release of the powdered agent and/or the pressurized fluid within enclosure 14.

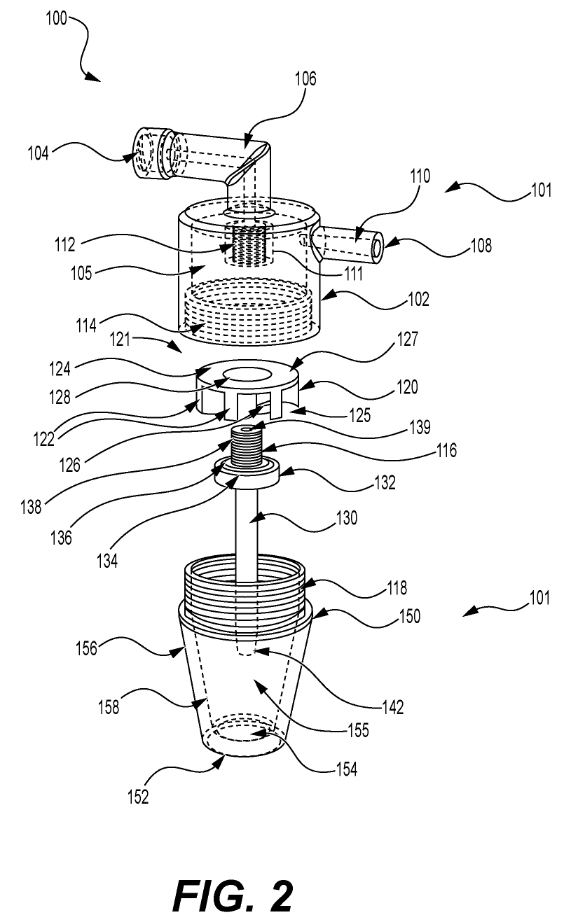

FIG. 2 shows an exemplary valve assembly 100, according to one or more embodiments. Valve assembly 100 may be part of delivery system 10 as discussed with reference to FIG. 1 above, such as disposed in enclosure 14. Valve assembly 100 may include a chamber and/or enclosure 101 for storing the agent, an agitation mechanism 120 rotatably disposed inside enclosure 101, and an outlet tube 130 disposed inside enclosure 101 and extending through agitation mechanism 120. A predetermined amount of agent (e.g., 3 mg to 5 mg) may be stored within enclosure 101 for delivery from valve assembly 100. The predetermined amount of agent stored therein may vary depending on a desired application of use, such as a treatment of a subject (e.g., patient) or a target treatment site. Enclosure 101 may include an upper body 102 and a lower body 150 that is removably coupled to upper body 102.

Upper body 102 may include an outlet 104, an outlet conduit 106 fluidly coupling an inner cavity 105 of upper body 102 to outlet 104 via a coupling mechanism 111, an inlet 108 positioned adjacent to outlet 104, and an inlet conduit 110 fluidly coupling inner cavity 105 to inlet 108. Upper body 102 may include a first fastening mechanism 112 within coupling mechanism 111 and a second fastening mechanism 114 disposed along a bottom portion of upper body 102, with each fastening mechanism 112, 114 being configured for removably coupling one or more components of valve assembly 100 to upper body 102. In the example, first fastening mechanism 112 may be disposed along an interior surface of coupling mechanism 111 and second fastening mechanism 114 may be disposed along an interior surface of upper body 102 that defines inner cavity 105. Coupling mechanism 111 may have a generally cylindrical body that extends downward from outlet conduit 106 adjacent to a top portion of upper body 102 and at least partially into inner cavity 105. In the example, coupling mechanism 111 may include a first diameter that is generally smaller than a second diameter of an opening formed in agitation mechanism 120, as further described below.

Still referring to FIG. 2, and as described above, outlet 104 may be in fluid communication with inner cavity 105 via outlet conduit 106, and inlet 108 may be in fluid communication with inner cavity 105via inlet conduit 110. Inlet 108 may be fluidly coupled to a fluid source (not shown), such as a pressurized medium source, and the fluid source may be configured to deliver a pressurized fluid (e.g., a gas) into enclosure 101 via inlet 108. Outlet 104 may be fluidly coupled to a catheter (not shown) to deliver a mixture of the pressurized fluid and agent out of enclosure 101. In the example, each of fastening mechanisms 112, 114 may include threaded portions that are configured to removably couple with other fastening mechanisms for securing coupling mechanism 111 and upper body 102 to one or more other components of valve assembly 100, respectively, as will be described in detail below.

Lower body 150 may include a bottom exterior surface 152, a bottom interior surface 154, a side exterior surface 156, and a side interior surface 158. Bottom interior surface 154 and side interior surface 158 may collectively define an inner cavity 155 of lower body 150. In the example, at least side interior 158 may be tapered radially inwards towards bottom interior surface 154. Lower body 150, and particularly inner cavity 155, may include a cyclonic configuration and/or cross-sectional profile that is at least partially defined by the tapered shape of side interior surface 158. In this instance, side interior surface 158 may be sized, shaped, and/or otherwise configured to guide movement of the pressurized fluid entering enclosure 101 via inlet 108 in a clockwise or counterclockwise rotative direction towards bottom interior surface 154.

As described herein, the cyclonic configuration and/or tapered cross-sectional profile of lower body 150, and particularly of inner cavity 155 as defined by side interior surface 158, may be configured to facilitate and/or enhance formation of a turbulent flow of pressurized fluid with the agent stored along bottom interior surface 154 to create a mixture of pressurized fluid and agent for delivery out of valve assembly 100. It should be appreciated that reference to a cyclonic configuration and/or cross-sectional profile of a surface or body throughout the present disclosure may correspond to a tapered, an angled, conical, and/or otherwise sloping shape. Lower body 150 may include a third fastening mechanism 118 disposed along an upper portion of side exterior surface 156. In the embodiment, third fastening mechanism 118 may include one or more threaded regions that are sized, shaped, and/or otherwise configured to threadably mate and/or couple with other fastening mechanisms of valve assembly 100.

For example, upper body 102 may be configured to rotatably couple with lower body 150 via an engagement between second fastening mechanism 114 and third fastening mechanism 118, as best seen in FIG. 4. In this instance, inner cavity 105 of upper body 102 may be in fluid communication with inner cavity 155 of lower body 150, and each of inner cavities 105, 155 may collectively define an enclosed cavity and/or volume of enclosure 101. Although fastening mechanisms 112, 114, 118 may be shown and described herein as threaded regions, it should be appreciated that various other suitable fastening mechanisms may be incorporated into valve assembly 100 without departing from a scope of this disclosure. For example, one or more of fastening mechanisms 112, 114, 118 may include, but are not limited to, one or more clamps, clips, tabs, protrusions, arms, etc.

Referring to FIGS. 2-5, agitation mechanism 120 may include a main body 121 defined between a top (closed) end 124 having a top planar surface 127, a bottom (open) end 125, and a plurality of blades 122 extending between top end 124 and bottom end 125. The plurality of blades 122 may extend radially outwards from main body 121. Agitation mechanism 120 may include a seat 126 positioned adjacent to bottom end 125 and an opening 128 extending through main body 121 between top end 124 and bottom end 125. In some embodiments, seat 126 may be positioned across and/or between each of the plurality of blades 122. In other words, seat 126 may be coupled to bottom end 125 opposite top planar surface 127. In some embodiments, seat 126 may extend along and/or define a lower portion of each of the plurality of blades 122 adjacent to bottom end 125.

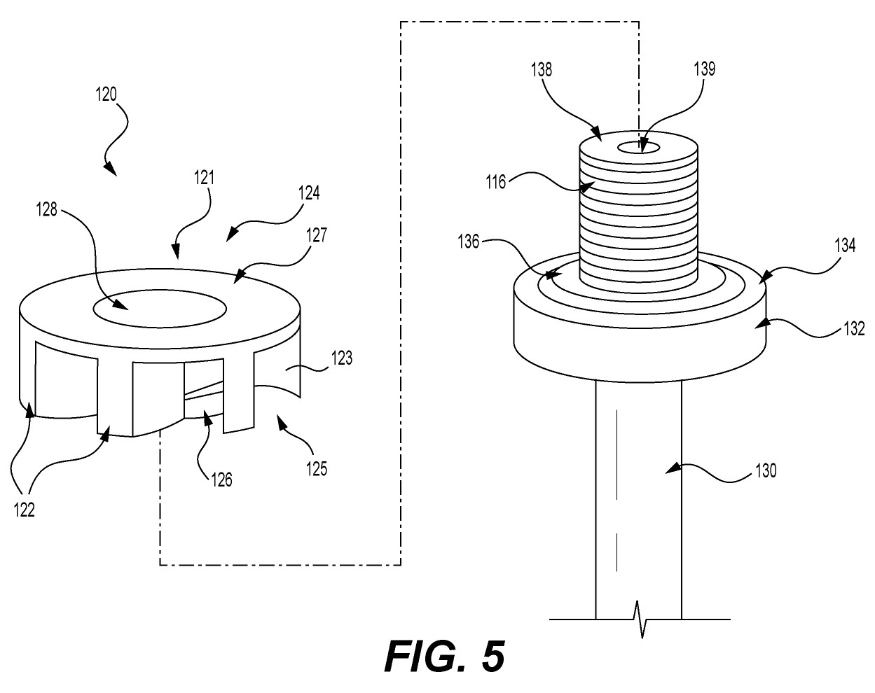

The plurality of blades 122 may extend radially outwards from main body 121, and may be disposed between top end 124 and bottom end 125 such that top planar surface 127 covers and/or conceals the plurality of blades 122 along top end 124 and bottom end 125 exposes the plurality of blades 122 to a surrounding environment of enclosure 101, and particularly inner cavities 105, 155. In some embodiments, as best seen in FIG. 5, the plurality of blades 122 may include a curved and/or angled configuration relative to top planar surface 127, such that each of the plurality of blades 122 may include a curvature and/or arc relative to the planar configuration of top planar surface 127. As described herein, a size, a shape, and/or a configuration of blades 122 may generate a turbulent flow of pressurized fluid and agent within enclosure 101 upon movement (e.g., rotation) of agitation mechanism 120 relative to upper body 102 and lower body 150.

Still referring to FIGS. 2-5, opening 128 may be disposed through a central axis of agitation mechanism 120, and particularly main body 121, by extending through top planar surface 127 towards bottom end 125. Stated differently, opening 128 may extend through an entirety of main body 121 between top end 124 and bottom end 125. As briefly described above, opening 128 may include a second diameter that is relatively greater than the first diameter of coupling mechanism 111. Accordingly, opening 128 may be sized, shaped, and/or otherwise configured to receive coupling mechanism 111 therein when agitation mechanism 120 is coupled to upper body 102.

As described in further detail herein, agitation mechanism 120 may be configured to guide, propel, and/or direct a pressurized fluid received within enclosure 101 from inlet 108 towards the agent stored within lower body 150 in response to rotating within upper body 102 about outlet tube 130 and coupling mechanism 111, thereby agitating the agent within enclosure 101. It should be appreciated that agitation mechanism 120 may be formed of various suitable materials, including but not limited to, metal, plastic (e.g., polyethylene), and more.

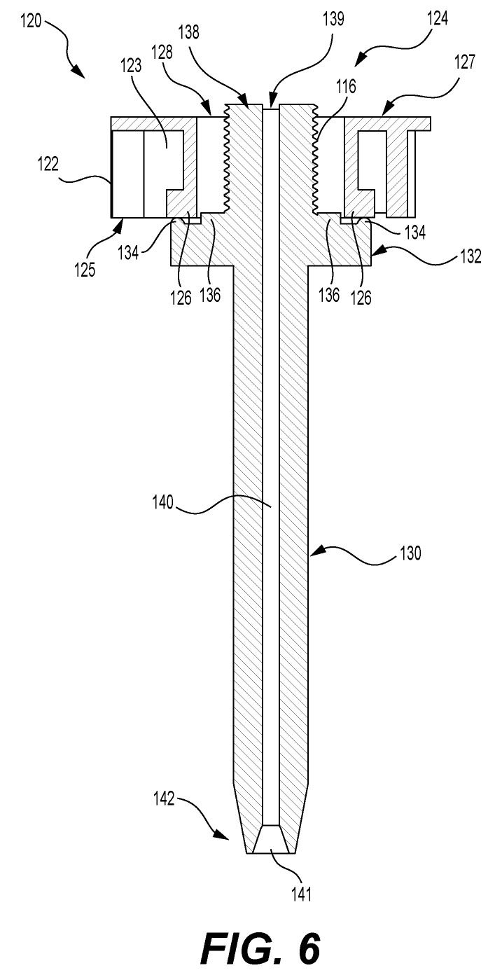

Still referring to FIGS. 2-5, outlet tube 130 may include a longitudinal length defined between a first end 138 and a second end 142 that is positioned along the longitudinal length of outlet tube 130 opposite to first end 138. As best seen in FIGS. 5-6, outlet tube 130 may include a platform 132 that extends radially outwards adjacent to first end 138, a ring 134 disposed about a boundary of platform 132 that protrudes outwardly in an upwards direction towards first end 138, and a step 136 disposed radially-inwards relative to ring 134 and extends outwardly from platform 132 in the upwards direction towards first end 138. In other words, ring 134 may define an outer lip extending along an outer perimeter of platform 132 and step 136 may define an inner ledge extending along an inner perimeter of platform 132.

Platform 132 may be configured to receive main body 121 when agitation mechanism 120 and outlet tube 130 are coupled to one another with first end 138 disposed within opening 128. It should be appreciated that first end 138 may include a third diameter that is relatively smaller than the second diameter of opening 128 such that first end 138 may be received within opening 128 when outlet tube 130 is coupled to actuation mechanism 120. Ring 134 may be sized, shaped, and/or otherwise configured to protrude in the upwards direction away from platform 132 and towards bottom end 125. As best seen in FIG. 6, ring 134 may be configured to interface with, and more particularly abut against or otherwise contact, seat 126 when outlet tube 130 is coupled to agitation mechanism 120. Step 136 may be sized, shaped, and/or otherwise configured to protrude in the upwards direction towards first end 138 and away from platform 132 for receipt within opening 128 when outlet tube 130 is coupled to agitation mechanism 120. In some embodiments, step 136 may be configured to contact an interior surface of main body 121 that defines opening 128 to support and/or stabilize the rotative motion of main body 121 about first end 138 when outlet tube 130 is coupled to agitation mechanism 120.

Referring back to FIG. 2, outlet tube 130 may include a fourth fastening mechanism 116 disposed about an exterior surface of first end 138. First end 138 may be sized, shaped, and/or otherwise configured to extend through opening 128 and couple with upper body 102 at coupling mechanism 111 via an engagement between first fastening mechanism 112 and fourth fastening mechanism 116. In this instance, with agitation mechanism 120 received on platform 132 and first end 138 threadably coupled to coupling mechanism 111, agitation mechanism 120 may be configured to receive coupling mechanism 111 within opening 128. Stated differently, as best seen in FIG. 4, coupling mechanism 111 may be disposed inside opening 128 when outlet tube 130 is coupled to upper body 102 via the engagement between first fastening mechanism 112 of coupling mechanism 111 and fourth fastening mechanism 118 of first end 138. In this instance, agitation mechanism 120 may be configured to move (e.g., rotate) about outlet tube 130 and coupling mechanism 111 with each of first end 138 and coupling mechanism 111 received within opening 128 and through main body 121.

Referring specifically to FIG. 6, outlet tube 130 may include a channel 140 having a longitudinal length defined between a first opening 139 at first end 138, and a second opening 141 at second end 142. In other words, the longitudinal length of channel 140 may correspond to the longitudinal length of outlet tube 130 defined between first end 138 and second end 142. With outlet tube 130 rotatably coupled to upper body 102 via the engagement between first fastening mechanism 112 and fourth fastening mechanism 116, channel 140 may be in fluid communication with outlet conduit 106 and outlet 104 via coupling mechanism 111.

As described herein, channel 140 may be configured to receive a mixture of pressurized fluid received in enclosure 101 via inlet 108 and the agent stored in enclosure 101 along bottom interior surface 154 at second opening 141, and direct the mixture through channel 140 for delivery towards outlet conduit 106 via first opening 139. For example, inlet 108 may be configured to direct the pressurized fluid into upper body 102 and towards agitation mechanism 120, thereby causing agitation mechanism 120 to rotate in response to the plurality of blades 122 capturing the pressurized fluid from inlet 108 to rotate main body 121 about first end 138. Agitation mechanism 120 may be configured to direct the pressurized fluid captured by the plurality of blades 122 downwards towards bottom interior surface 154, thereby creating a cyclonic motion of pressurized fluid. The cyclonic motion of pressurized fluid may be at least partially formed in response to the curvature of blades 122 or the cyclonic (tapered) configuration of inner cavities 105, 155.

Stated differently, agitation mechanism 120 may be configured to generate and/or increase a pressure differential within enclosure 101 to strengthen a vortex of turbulent flow formed therein for stirring the agent with increased velocity. It should be appreciated that reference to a cyclonic motion of pressurized fluid, agent, or a mixture thereof throughout the present disclosure may correspond to generating and/or increasing a pressure within enclosure 101, a vortex of turbulent flow within enclosure 101, and/or a velocity or speed of movement of the aforementioned materials within enclosure 101 for agitating (e.g., fluidizing) the agent with pressurized fluid.

In the embodiment, the plurality of blades 122 may be configured to receive and propel the pressurized fluid towards side interior surface 158 to create a turbulent flow directed towards the agent stored along bottom interior surface 154. In other words, agitation mechanism 120 may be configured to push the pressurized fluid relatively downwards from upper body 102 to lower body 150, and particularly towards bottom interior surface 154 on which the agent is stored. It should be appreciated that the cyclonic motion of the pressurized fluid created by agitation mechanism 120 within lower body 150 may include moving the pressurized fluid in a clockwise or counterclockwise rotative direction towards bottom interior surface 154. The cyclonic motion of pressurized fluid may include, but is not limited to, a circular movement, a twisting movement, a swirling movement, a rotative movement, a funnel-like movement, and/or a spiral movement within inner cavity 155.

In addition, upper body 102 may be sized, shaped, and/or otherwise configured to include a cyclonic and/or tapered configuration to guide movement of the pressurized fluid received from inlet 108 towards lower body 150 where the agent is stored. The agent stored within enclosure 101 is agitated in response to agitation mechanism 120 causing the pressurized fluid to move downwards into lower body 150 in the cyclonic motion within enclosure 101. In response to agitating the agent, outlet tube 130 may be configured to receive the mixture of the pressurized fluid and the agent via second opening 141 at second end 142, and direct the mixture upwards through channel 140 for delivery out of first opening 139 and into outlet 104 that is fluidly coupled to first opening 139 via outlet conduit 106. In other words, channel 140 may be configured to guide the mixture through first opening 139 and towards outlet conduit 106, thereby allowing for fluid communication between outlet tube 130 and outlet 104 for delivering the mixture out of enclosure 101.

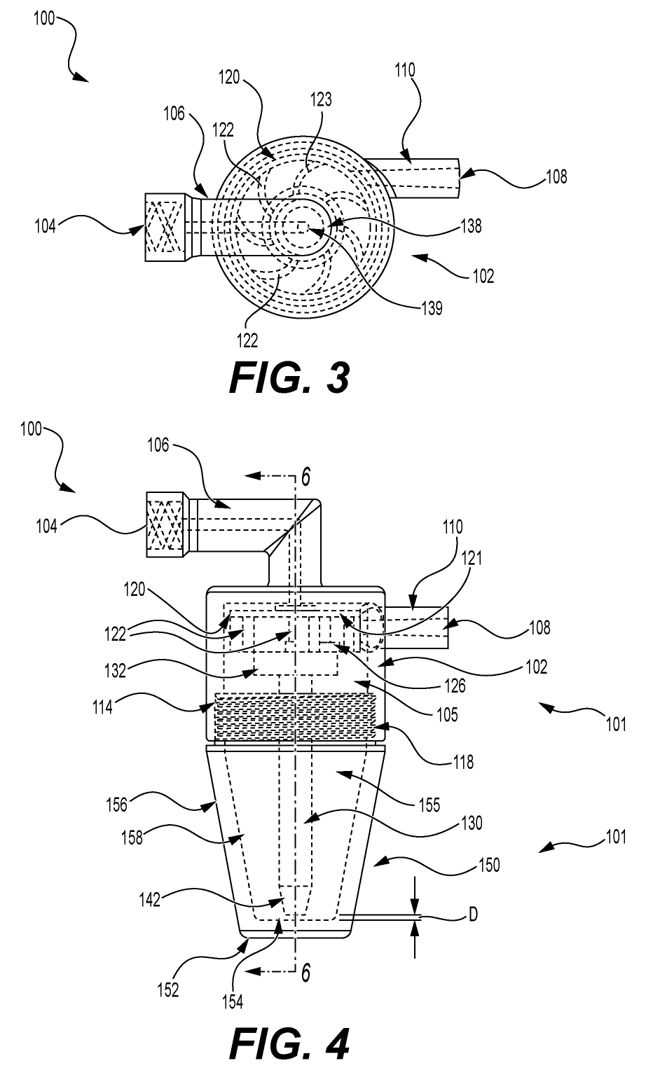

Referring back to FIGS. 3-4, agitation mechanism 120 may be sized, shaped, and/or otherwise configured as a rotatable propeller with each of the plurality of blades 122 extending downwards from top planar surface 127. In the example, each of the plurality of blades 122 may include an interior surface 123 that is curved, thereby defining an arc configuration of blades 122. Interior surfaces 123 may be configured to capture the pressurized fluid received within enclosure 101 via inlet 108 to propel the plurality of blades 122 in a rotatable movement within upper body 102. In some embodiments, inlet 108 is arranged, oriented, and/or angled relative to agitation mechanism 120 such that the pressurized fluid delivered into enclosure 101 by inlet conduit 110 may contact interior surfaces 123 of blades 122 at a suitable angle to maximize a rotative force generated against blades 122. With main body 121 closed off along top end 124 by top planar surface 127 and open along bottom end 125, the plurality of blades 122 may be configured to direct the pressurized fluid received by interior surfaces 123 downwards through bottom end 125 and towards the agent stored in lower body 150.

In the example, agitation mechanism 120 may be positioned within upper body 102 such that the plurality of blades 122 are arranged with interior surface 123 of one or more blades 122 facing inlet conduit 110. Given the curved and/or arc configuration of blades 122, it should be appreciated that interior surfaces 123 may be generally curved and/or angled. In this instance, the pressurized fluid received in enclosure 101 via inlet 108 is directed against interior surfaces 123, such that blades 122 may be configured to capture the pressurized fluid across interior surfaces 123. In other words, upon receipt of the pressurized fluid into enclosure 101 through inlet 108, agitation mechanism 120 may be configured to capture the pressurized fluid along interior surfaces 123 of the one or more blades 122 facing inlet conduit 110, thereby causing the plurality of blades 122 to rotate main body 121 about outlet tube 130.

Referring now to FIGS. 5-6, which omit upper body 102 and lower body 150 for clarity purposes to clearly depict the design of agitation mechanism 120 and outlet tube 130, first end 138 may extend through agitation mechanism 120 via opening 128 to couple with coupling mechanism 111 as described in detail above. Coupling mechanism 111 (see FIG. 2), when received in opening 128, may be in contact with step 136. Ring 134 may be configured to receive and/or contact seat 126 when outlet tube 130 is coupled to coupling mechanism 111 and received through agitation mechanism 120. Ring 134 may be sized, shaped, and/or otherwise configured to abut against seat 126 to raise main body 121 off of platform 132. In this instance, ring 134 may be configured to minimize a surface area of outlet tube 130 that abuts against agitation mechanism 120, thereby reducing frictional resistance between agitation mechanism 120 and outlet tube 130 during rotation of main body 121 about first end 138.

Stated differently, ring 134 may be configured to provide a relatively smaller diameter and/or surface area relative to platform 132 for contacting seat 126 such that friction between ring 134 and seat 126 is minimized as agitation mechanism 120 rotates about outlet tube 130. The reduction of friction between seat 126 and ring 134 provides efficient rotation of agitation mechanism 120 around outlet tube 130. As described, agitation mechanism 120 may be configured to agitate the agent stored in lower body 150 in response to main body 121 rotating about first end 138 to create a turbulent flow of pressurized fluid within enclosure 101.

In this instance, agitation mechanism 120 may be configured to direct the pressurized fluid downwards from upper body 102 into lower body 150 in the cyclonic motion towards bottom interior surface 154 to agitate the agent stored therein. In some embodiments, the cyclonic motion of pressurized fluid generated by agitation mechanism 120 may combine the pressurized fluid with the agent, such that valve assembly 100 may be configured to create a mixture of the pressurized fluid and agent. Outlet tube 130 may be configured to receive the mixture into second opening 141 at second end 142. Lower body 150 and/or outlet tube 130 may be sized, shaped, and/or otherwise configured to form a gap D between second end 142 and bottom interior surface 154 when upper body 102, outlet tube 130, and lower body 150 are coupled to one another. For example, outlet tube 130 may have a longitudinal length that positions second end 142 adjacent to bottom interior surface 154, with gap D defining an offset distance disposed therebetween.

With valve assembly 100 including gap D disposed between second end 142 and bottom interior surface 154, outlet tube 130 may be configured to receive and direct the mixture of pressurized fluid and agent through channel 140 and towards outlet 104. It should be appreciated that gap D may include various suitable dimensions without departing from a scope of this disclosure. In some examples, gap D may range from approximately 1 millimeter (mm) to 10 mm. For example, gap D may range from approximately 1 mm to 2 mm. It should be appreciated that as gap D between second end 142 and bottom interior surface 154 increases, the amount of mixture of pressurized fluid and agent received in second opening 141 may increase proportionately. On the other hand, it should be understood that decreasing gap D may reduce the amount of the mixture of pressurized fluid and agent received in second opening 141 and delivered through channel 140 towards outlet 104. Accordingly, adjusting a size and/or shape of outlet tube 130 and/or lower body 150 may correspond to a modified gap D, which may define a corresponding amount of the mixture delivered from valve assembly 100.

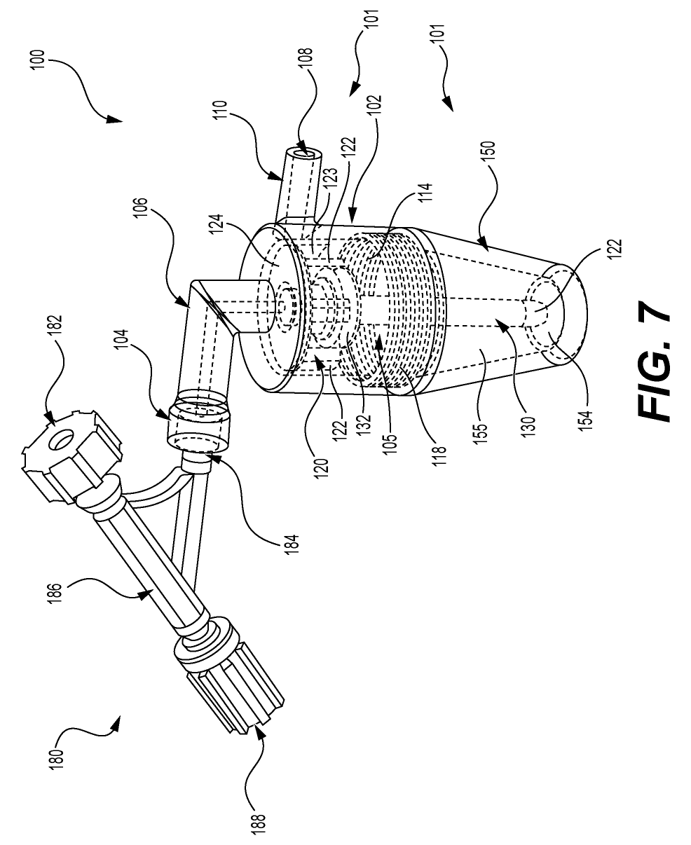

FIG. 7 shows valve assembly 100 coupled to an exemplary connector assembly 180 at outlet 104. Connector assembly 180 may include a port 182, an inlet 184 configured to fluidly couple connector assembly 180 to outlet 104, an outlet 188, and a passage 186 extending between and in fluid communication with each of port 182, inlet 184, and outlet 188. Connector assembly 180 may be removably coupled to valve assembly 100 via connection between inlet 184 and outlet 104, such that the mixture of pressurized fluid and agent delivered out of valve assembly 100 from outlet 104 may be received by connector assembly 180 via inlet 184.

Port 182 may be sized, shaped, and/or otherwise configured to receive a tool and/or an instrument therethrough (e.g., a guidewire). Passage 186 may have a longitudinal length defined between port 182 and outlet 188 that includes a lumen for fluidly coupling port 182 to outlet 188. Passage 186 may be configured to receive and guide the tool or instrument (not shown) received at port 182 through the lumen and towards outlet 188, the tool serving to facilitate clearing (e.g., de-clogging) any residual amount of agent delivered out of valve assembly 100 and remaining inside connector assembly 180 to prevent clogging. In other embodiments, the tool may provide a mechanical means for removing moisture that may lead to clogging by pushing the tool therethrough.

In some embodiments, outlet 188 may be removably coupled to a catheter or another device connected to valve assembly 100 and/or delivery device 10, such as catheter 36 (see FIG. 1). In this instance, connector assembly 180 may be directly coupled to the delivery conduit and configured to guide the tool or instrument into the delivery conduit to clear residual amounts of agent remaining within the delivery conduit. Although not shown, in other embodiments connector assembly 180 may include an actuator (e.g., a depressible button) at port 182 that is operable to automatically move (e.g., translate) a tool or instrument that is predisposed inside passage 186. For example, the actuator may be biased by a spring and the tool or instrument is mounted within passage 186 and coupled to the spring.

Still referring to FIG. 7, port 182 may include a seal (not shown) that is configured to allow the tool or instrument to enter into port 182 without the release of any pressurized fluid, agent, or a mixture thereof out of connector assembly 180 via port 182. In other words, the seal at port 182 may inhibit fluid communication from passage 186 to an exterior of connector assembly 180. Port 182 may be configured to allow the tool or instrument (not shown) to extend through passage 186 and towards outlet 188.

In exemplary use, valve assembly 100 may be configured to receive the pressurized fluid within enclosure 101 via inlet 108 from the fluid source (not shown), such as in response to actuation of actuation mechanism 30 of delivery device 10 (see FIG. 1). Referring to FIGS. 3-4, agitation mechanism 120 may be configured to rotate within upper body 102 and about outlet tube 130 based on the force exerted against the plurality of blades 122, and particularly interior surfaces 123, from the pressurized fluid received within enclosure 101.

Agitation mechanism 120 may be configured to direct the pressurized fluid received against interior surfaces 123 of blades 122 downwards from upper body 102 towards lower body 150. With main body 121 being closed along top end 124 by top planar surface 127 and open along bottom end 125, the plurality of blades 122 may be configured to push the pressurized fluid downwards towards bottom interior surface 154 that the agent is stored on in a cyclonic motion due to the cross-sectional profile and/or configuration of enclosure 101, and specifically the interior cavities of upper body 102 and lower body 150. The pressurized fluid received in enclosure 101 may combine with the agent (e.g., hemostatic powder) stored in lower body 150 based on the cyclonic motion of the pressurized fluid generated within enclosure 101 by agitation mechanism 120.

Valve assembly 100, and particularly interior surfaces 154, 158 of lower body 150, may be configured to further direct the mixture of pressurized fluid and agent generated within lower body 150 by agitation mechanism 120 towards second opening 141 of outlet tube 130 (see FIG. 6). Stated differently, a size, a shape, and/or a configuration of interior surfaces 154, 158 may be configured to guide the mixture towards second end 142 for receipt within second opening 141 due to gap D disposed between bottom interior surface 154 and second end 142 (see FIG. 4). Upon entry into second opening 141, outlet tube 130 may be configured to guide the mixture of the pressurized fluid and the agent through channel 140 and towards outlet 104 that is in fluid communication with first opening 139 via coupling mechanism 111. In embodiments where valve assembly 100 is coupled to connector assembly 180, outlet 104 may be configured to direct the mixture into inlet 184 prior to delivery through a delivery conduit (e.g. catheter 36).

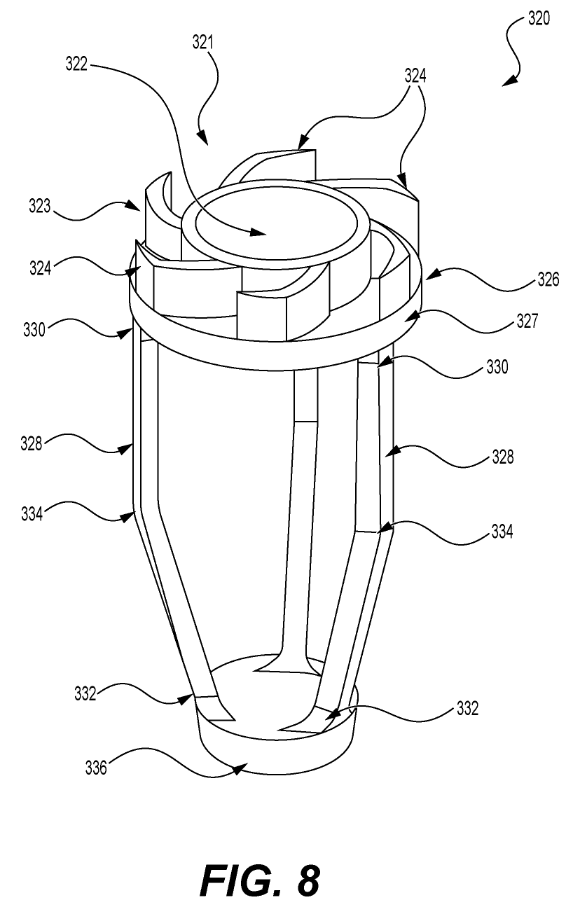

FIG. 8 shows another exemplary agitation mechanism 320 of valve assembly 100. It should be appreciated that agitation mechanism 320 may be configured and operable in a substantially similar manner as agitation mechanism 120 shown and described above, such that valve assembly 100 may include agitation mechanism 320 in place of agitation mechanism 120 without departing from a scope of this disclosure. Agitation mechanism 320 may include substantially similar features as agitation mechanism 120 except for the differences explicitly described herein.

For example, agitation mechanism 320 may include a main body 321 having a top end 323, a bottom end 326 opposite top end 323, and a plurality of blades 324 disposed between top end 323 and bottom end 323. Agitation mechanism 320 may be sized, shaped, and/or otherwise configured as a rotatable propeller or turbine, and the plurality of blades 324 may extend radially outwards from main body 321. In the embodiment, main body 321 may be open along top end 323 and closed along bottom end 326 by a bottom planar surface 327. In some embodiments, the plurality of blades 324 may include a curved and/or angled configuration relative to bottom planar surface 327, such that each of the plurality of blades 324 may include a curvature and/or arc relative to the planar configuration of bottom planar surface 327. As described herein, a size, shape, and/or configuration of blades 324 may generate a turbulent flow of pressurized fluid and agent within enclosure 101 upon movement (e.g., rotation) of agitation mechanism 320.

Still referring to FIG. 8, main body 321 may include an opening 322 extending through a central axis of main body 321 through top end 323 and bottom end 326, with the plurality of blades 324 disposed about and/or extending radially outwards from opening 322. Stated differently, main body 321 may be configured such that the plurality of blades 324 are exposed along top end 323 and covered along bottom end 326 by bottom planar surface 327. Opening 322 may be sized, shaped, and/or otherwise configured to receive first end 138 of outlet tube 130 and coupling mechanism 111 of upper body 102 similar to opening 128 shown and described above (see FIG. 6).

Agitation mechanism 320 may include a plurality of arms 328 extending relatively downwards from bottom planar surface 327 towards a base 336 disposed at a terminal end of the plurality of arms 328. It should be appreciated that base 336 may define a secondary body of agitation mechanism 320, and may be positioned relatively below main body 321 such that second end 142 of outlet tube 130 is suspended relatively above base 336 when outlet tube 130 is coupled to agitation mechanism 320 with first end 138 received within opening 322. In this instance, second end 142 and base 336 may form gap D therebetween to facilitate receipt of the mixture of pressurized fluid and agent into outlet tube 130 via second opening 141.

Still referring to FIG. 8, each of the plurality of arms 328 may include a longitudinal length defined between an upper end 330 that is attached to bottom planar surface 327 and a lower end 332 that is positioned opposite to upper end 330 and attached to base 336. The plurality of arms 328 may be sized and/or shaped such that at least a lower portion of arms 328 (e.g., lower ends 332) may extend into and/or be disposed inside lower body 150 as an upper portion of arms 328 (e.g., upper ends 330) is disposed inside upper body 102. In this instance, main body 321 and base 336 are separated from one another within upper body 102 and lower body 150, respectively, by a longitudinal length of the plurality of arms 328.

Each of the plurality of arms 328 may further include a joint 334 disposed between upper end 330 and lower end 332, such that the plurality of arms 328 may have a non-linear and/or angled configuration between upper end 330 and lower end 332. Joint 334 may be sized, shaped, and/or otherwise configured to conform a cross-sectional profile of the plurality of arms 328 to a corresponding cross-sectional profile of lower body 150. Although the plurality of arms 328 are shown and described herein as including a single joint 334 between upper end 330 and lower end 332, it should be appreciated that one or more of the plurality of arms 328 may include additional and/or fewer joints 334 without departing from a scope of this disclosure. The plurality of arms 328 may be configured to extend from bottom planar surface 327 to base 336 to agitate the agent stored within enclosure 101 upon rotation of agitation mechanism 320. In other words, the plurality of arms 328 may be configured to abut against and/or contact the agent, thereby moving the agent from a compacted state to a fluidized / agitated state prior to delivery out of enclosure 101 via outlet tube 130.

In exemplary use, the pressurized fluid received in enclosure 101 from the fluid source (not shown) via inlet 108 may contact agitation mechanism 320, and particularly the plurality of blades 324, causing the plurality of blades 324 to rotate about outlet tube 130 within enclosure 101. Agitation mechanism 320 may be configured to direct the pressurized fluid received within enclosure 101 downwards through upper body 102 and into lower body 150 towards bottom interior surface 154 in a cyclonic motion, as described in detail above. In the embodiment, one or more of the plurality of blades 324 and/or the plurality of arms 328 may be configured to generate and/or enhance the cyclonic motion of pressurized fluid within enclosure 101. The cyclonic motion of pressurized fluid generated by agitation mechanism 320 may agitate the agent stored within enclosure 101, thereby combining the agent and pressurized fluid within enclosure 101 prior to delivery via outlet tube 130.

In addition, the plurality of arms 328 may be configured to push, abut against, contact, and/or otherwise unsettle the agent that may be settled in a compact state within lower body 150. Accordingly, the plurality of arms 328 may be configured to agitate the agent within enclosure 101 and/or combine the agent with the pressurized fluid by rotating within enclosure 101. As the mixture of agent and pressurized fluid moves within enclosure 101, outlet tube 130 may be configured to receive the mixture at second end 142 and through channel 140 for delivery towards outlet 104 from valve assembly 100.

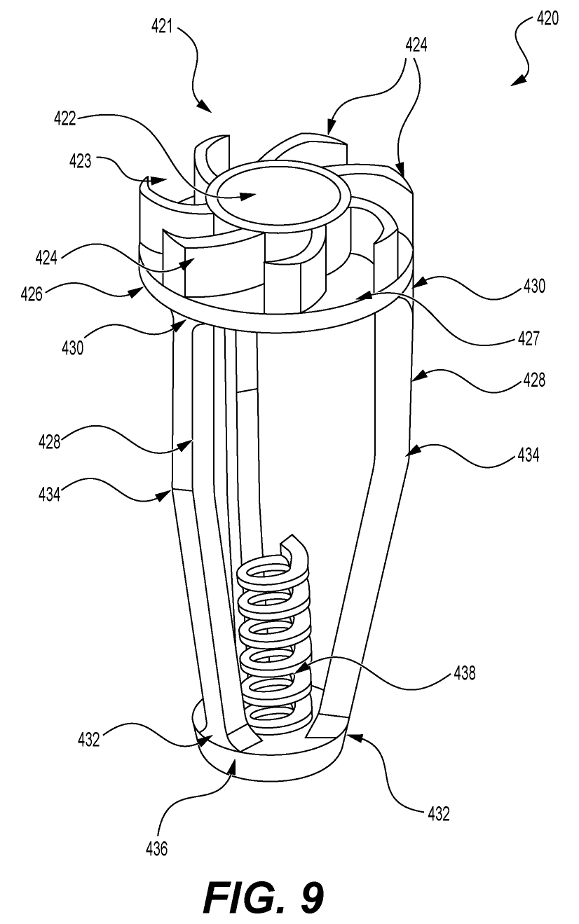

FIG. 9 shows another exemplary agitation mechanism 420 of the valve assembly 100. It should be appreciated that agitation mechanism 420 may be configured and operable in a substantially similar manner as agitation mechanism 120 shown and described above, such that valve assembly 100 may include agitation mechanism 420 in place of agitation mechanism 120 (see FIGS. 2-7) without departing from a scope of this disclosure. Agitation mechanism 420 may include substantially similar features as agitation mechanism 120 except for the differences explicitly described herein.

For example, agitation mechanism 420 may include a main body 421 having a top end 423, a bottom end 426 opposite top end 423, and a plurality of blades 424 disposed between top end 423 and bottom end 423. Agitation mechanism 420 may be sized, shaped, and/or otherwise configured as a rotatable propeller, and the plurality of blades 424 may extend radially outwards from main body 421. In the embodiment, main body 421 may be open along top end 423 and closed along bottom end 426 by a bottom planar surface 427. In some embodiments, the plurality of blades 424 may include a curved and/or angled configuration relative to bottom planar surface 427, such that each of the plurality of blades 424 may include a curvature and/or arc relative to the planar configuration of bottom planar surface 427. As described herein, a size, shape, and/or configuration of blades 424 may generate a turbulent flow of pressurized fluid and agent within enclosure 101 upon movement (e.g., rotation) of agitation mechanism 420.

Still referring to FIG. 9, main body 421 may include an opening 422 extending through a central axis of main body 421 through top end 423 and bottom end 426, with the plurality of blades 424 disposed about and/or extending radially outwards from opening 422. Stated differently, main body 421 may be configured such that the plurality of blades 424 are exposed along top end 423 and covered along bottom end 426 by bottom planar surface 427. Opening 422 may be sized, shaped, and/or otherwise configured to receive first end 138 of outlet tube 130 and coupling mechanism 111 of upper body 102 similar to opening 128 shown and described above (see FIG. 6).

Agitation mechanism 420 may include a plurality of arms 428 extending relatively downwards from bottom planar surface 427 towards a base 436 disposed at a terminal end of the plurality of arms 428. It should be appreciated that base 436 may define a secondary body of agitation mechanism 420, and may be positioned relatively below main body 421 such that second end 142 of outlet tube 130 is suspended relatively above base 436 when outlet tube 130 is coupled to agitation mechanism 420 with first end 138 received within opening 422. In this instance, second end 142 and base 436 may form gap D therebetween to facilitate receipt of the mixture of pressurized fluid and agent into outlet tube 130 via second opening 141.

Still referring to FIG. 9, each of the plurality of arms 428 may include a longitudinal length defined between an upper end 430 that is attached to bottom planar surface 427 and a lower end 432 that is positioned opposite to upper end 430 and attached to base 436. The plurality of arms 428 may be sized and/or shaped such that at least a lower portion of arms 428 may extend into and/or be disposed inside lower body 150 as an upper portion of arms 428 is disposed inside upper body 102. In this instance, main body 421 and base 436 are separated from one another within upper body 102 and lower body 150, respectively, by a longitudinal length of the plurality of arms 428.

Each of the plurality of arms 428 may further include a joint 434 disposed between upper end 430 and lower end 432, such that the plurality of arms 428 may have a non-linear and/or angled configuration between upper end 430 and lower end 432. Joint 434 may be sized, shaped, and/or otherwise configured to conform a cross-sectional profile of the plurality of arms 428 to a corresponding cross-sectional profile of lower body 150. Although the plurality of arms 428 are shown and described herein as including a single joint 434 between upper end 430 and lower end 432, it should be appreciated that one or more of the plurality of arms 428 may include additional and/or fewer joints 434 without departing from a scope of this disclosure. The plurality of arms 428 may be configured to extend from bottom planar surface 427 to base 436 to agitate the agent stored within enclosure 101 upon rotation of agitation mechanism 420. In other words, the plurality of arms 428 may be configured to abut against and/or contact the agent, thereby moving the agent from a compacted state to a fluidized / agitated state prior to delivery out of enclosure 101 via outlet tube 130.

Agitation mechanism 420 may further include an agitator 438 disposed along an inner surface of base 436, with agitator 438 extending relatively upwards from base 436 towards main body 421. Agitator 438 may be disposed along a central portion of the inner surface on base 436 such that agitator 438 is positioned relatively inwards from the plurality of arms 428 disposed about an outer portion of base 436. In this instance, the plurality of arms 428 are positioned about agitator 438. Agitator 438 may include a longitudinal length defined between base 436 and main body 421, such that the longitudinal length of agitator 438 is relatively less than a longitudinal length of the plurality of arms 428 attached to and extending between base 436 and main body 421. In some embodiments, agitator 438 may include a coil, a screw, a prong, a protrusion, a projection, and/or various other mechanisms suitable for physically interfacing with the agent within enclosure 101.

In the example, agitator 438 may include a helical configuration that is configured to mix, move, and/or otherwise contact the agent to create a mixture of pressurized fluid and agent within enclosure 101. Agitator 438 may be further configured to lift and/or move the agent towards outlet tube 130, and particularly second end 142 for receipt within second opening 141. The longitudinal length of agitator 438 may at least partially define gap D between second end 142 of outlet tube to ensure the mixture of pressurized fluid and agent enters channel 140 during delivery from valve assembly 100. In addition, agitator 438 may be sized, shaped, and/or otherwise configured to direct the mixture into second end 142 and through channel 140 for delivery towards outlet 104 of enclosure 101 for delivery. As described herein, a size, a shape, and/or a structural configuration of agitator 438 may generate a directional flow of the mixture towards outlet tube 130 upon movement (e.g., rotation) of agitation mechanism 420.

In exemplary use, the pressurized fluid received in enclosure 101 from the fluid source (not shown) via inlet 108 may contact agitation mechanism 420, and particularly the plurality of blades 424, causing the plurality of blades 424 to rotate about outlet tube 130 within enclosure 101. Agitation mechanism 420 may be configured to direct the pressurized fluid received within enclosure 101 downwards through upper body 102 and into lower body 150 towards bottom interior surface 154 in a cyclonic motion, as described in detail above. In the embodiment, one or more of the plurality of blades 424, the plurality of arms 428, and/or the agitator 438 may be configured to generate and/or enhance the cyclonic motion of pressurized fluid within enclosure 101. The cyclonic motion of pressurized fluid generated by agitation mechanism 420 may agitate the agent stored within enclosure 101, thereby combining the agent and pressurized fluid within enclosure 101 prior to delivery via outlet tube 130.

In addition, each of the plurality of arms 428 and agitator 438 may be configured to push, abut against, contact, and/or otherwise unsettle the agent that may be settled in a compact state within lower body 150. Accordingly, the plurality of arms 428 and agitator 438 may be configured to agitate the agent within enclosure 101 and/or combine the agent with the pressurized fluid by rotating within enclosure 101. As the mixture of agent and pressurized fluid moves within enclosure 101, outlet tube 130 may be configured to receive the mixture at second end 142 and through channel 140 for delivery towards outlet 104 for delivery from valve assembly 100.

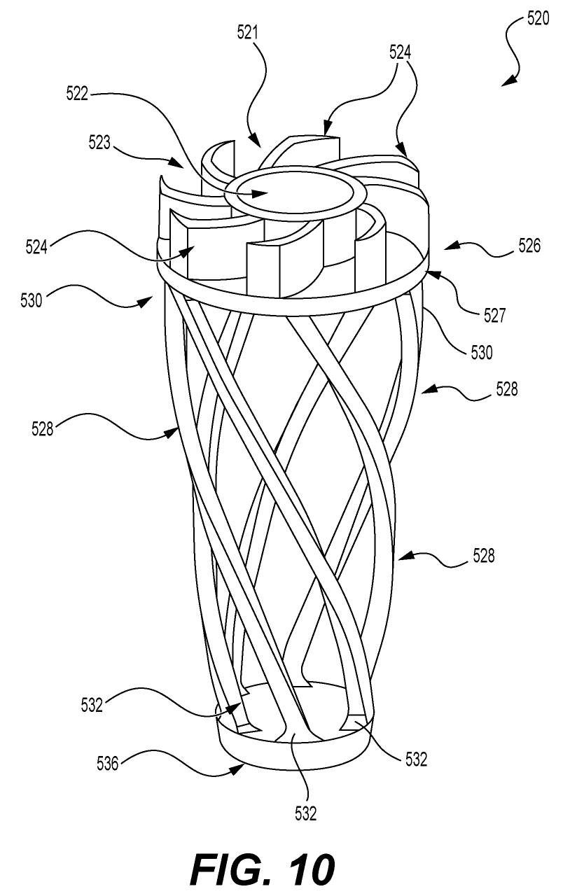

FIG. 10 shows another exemplary agitation mechanism 520 of valve assembly 100. It should be appreciated that agitation mechanism 520 may be configured and operable in a substantially similar manner as agitation mechanism 120 shown and described above, such that valve assembly 100 may include agitation mechanism 520 in place of agitation mechanism 120 without departing from a scope of this disclosure. Agitation mechanism 520 may include substantially similar features as agitation mechanism 120 except for the differences explicitly described herein.

For example, agitation mechanism 520 may include a main body 521 having a top end 523, a bottom end 526 opposite top end 523, and a plurality of blades 524 disposed between top end 523 and bottom end 526. Agitation mechanism 520 may be sized, shaped, and/or otherwise configured as a rotatable propeller, and the plurality of blades 524 may extend radially outwards from main body 521. In the embodiment, main body 521 may be open along top end 523 and closed along bottom end 526 by a bottom planar surface 527. In some embodiments, the plurality of blades 524 may include a curved and/or angled configuration relative to bottom planar surface 527, such that each of the plurality of blades 524 may include a curvature and/or arc relative to the planar configuration of bottom planar surface 527. As described herein, a size, shape, and/or configuration of blades 524 may generate a turbulent flow of pressurized fluid and agent within enclosure 101 upon movement (e.g., rotation) of agitation mechanism 520.

Still referring to FIG. 10, main body 521 may include an opening 522 extending through a central axis of main body 521 through top end 523 and bottom end 526, with the plurality of blades 524 disposed about and/or extending radially outwards from opening 522. Stated differently, main body 521 may be configured such that the plurality of blades 524 are exposed along top end 523 and covered along bottom end 526 by bottom planar surface 527. Opening 522 may be sized, shaped, and/or otherwise configured to receive first end 138 of outlet tube 130 and coupling mechanism 111 of upper body 102 similar to opening 128 shown and described above (see FIG. 6).

Agitation mechanism 520 may include a plurality of arms 528 extending relatively downwards from bottom planar surface 527 towards a base 536 disposed at a terminal end of the plurality of arms 528. It should be appreciated that base 536 may define a secondary body of agitation mechanism 520, and may be positioned relatively below main body 521 such that second end 142 of outlet tube 130 is suspended relatively above base 536 when outlet tube 130 is coupled to agitation mechanism 520 with first end 138 received within opening 522. In this instance, second end 142 and base 536 may form gap D therebetween to facilitate receipt of the mixture of pressurized fluid and agent into outlet tube 130 via second opening 141.

Still referring to FIG. 10, each of the plurality of arms 528 may include a longitudinal length defined between an upper end 530 that is attached to bottom planar surface 527 and a lower end 532 that is positioned opposite to upper end 530 and attached to base 536. The plurality of arms 528 may be sized and/or shaped such that at least a lower portion of arms 528 (e.g., lower ends 532) may extend into and/or be disposed inside lower body 150 as an upper portion of arms 528 (e.g., upper ends 530) is disposed inside upper body 102. In this instance, main body 521 and base 536 are separated from one another within upper body 102 and lower body 150, respectively, by a longitudinal length of the plurality of arms 528.

Each of the plurality of arms 528 may include a helical configuration extending from upper end 530 that is attached to bottom planar surface 527 to lower end 532 attached to base 536, such that the plurality of arms 528 may have a non-linear configuration between upper end 530 and lower end 532. The plurality of arms 528 may be configured to extend from bottom planar surface 527 to base 536 to agitate the agent stored within enclosure 101 upon rotation of agitation mechanism 520. In other words, the plurality of arms 528 may be configured to abut against and/or contact the agent, thereby moving the agent from a compacted state to a fluidized / agitated state prior to delivery out of enclosure 101 via outlet tube 130. In some embodiments, the plurality of arms 528 may be configured to lift and/or move the agent upwards towards outlet tube 130, and particularly second end 142, for receipt within second opening 141.

In exemplary use, the pressurized fluid received in enclosure 101 from the fluid source (not shown) via inlet 108 may contact agitation mechanism 520, and particularly the plurality of blades 524, causing the plurality of blades 524 to rotate about outlet tube 130 within enclosure 101. Agitation mechanism 520 may be configured to direct the pressurized fluid received within enclosure 101 downwards through upper body 102 and into lower body 150 towards bottom interior surface 154 in a cyclonic motion, as described in detail above. In the embodiment, one or more of the plurality of blades 524 and/or the plurality of arms 528 may be configured to generate and/or enhance the cyclonic motion of pressurized fluid within enclosure 101.The cyclonic motion of pressurized fluid generated by agitation mechanism 520 may agitate the agent stored within enclosure 101, thereby combining the agent and pressurized fluid within enclosure 101 prior to delivery via outlet tube 130.

In addition, the plurality of arms 528 may be configured to push, abut against, contact, and/or otherwise unsettle the agent that may be settled in a compact state within lower body 150. Accordingly, the plurality of arms 528 may be configured to agitate the agent within enclosure 101 and/or combine the agent with the pressurized fluid by rotating within enclosure 101. As the mixture of agent and pressurized fluid moves within enclosure 101, outlet tube 130 may be configured to receive the mixture at second end 142 and through channel 140 for delivery towards outlet 104 from valve assembly 100.

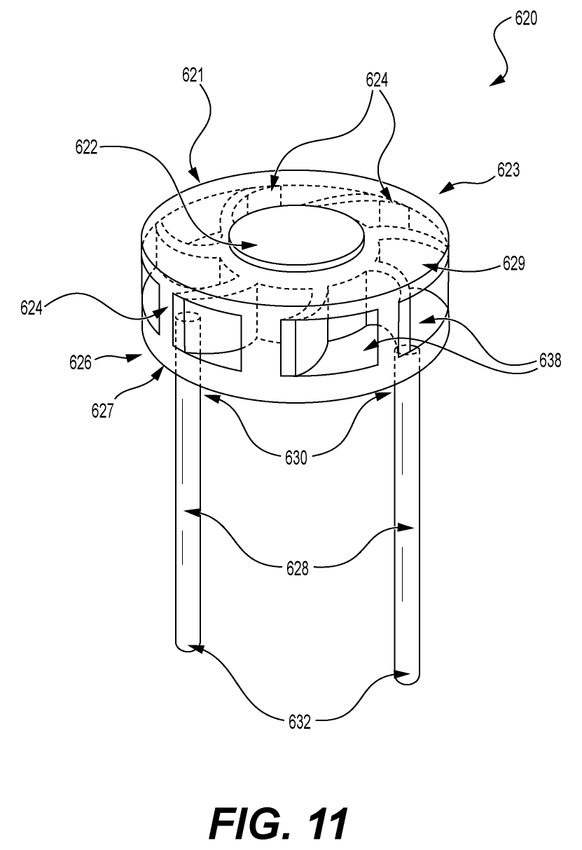

FIG. 11 shows another exemplary agitation mechanism 620 of the valve assembly 100. It should be appreciated that agitation mechanism 620 may be configured and operable in a substantially similar manner as agitation mechanism 120 shown and described above, such that valve assembly 100 may include agitation mechanism 620 in place of agitation mechanism 120 without departing from a scope of this disclosure. Agitation mechanism 620 may include substantially similar features as agitation mechanism 120 except for the differences explicitly described herein.

For example, agitation mechanism 620 may include a main body 621 having a top end 623, a bottom end 626 opposite top end 623, and a plurality of blades 624 disposed between top end 623 and bottom end 626. Agitation mechanism 620 may be sized, shaped, and/or otherwise configured as a rotatable propeller or turbine, and the plurality of blades 624 may extend radially outwards from main body 621. In the embodiment, main body 621 may be closed along each of top end 623 by a top planar surface 629 and bottom end 626 by a bottom planar surface 627. In some embodiments, the plurality of blades 624 may include a curved and/or angled configuration relative to top planar surface 629 and bottom planar surface 627, such that each of the plurality of blades 624 may include a curvature and/or arc relative to the planar configurations of planar surface 627, 629. As described herein, a size, shape, and/or configuration of blades 624 may generate a turbulent flow of pressurized fluid and agent within enclosure 101 upon movement (e.g., rotation) of agitation mechanism 620.

Still referring to FIG. 11, agitation mechanism 620 may include an opening 622 extending through a central axis of main body 621 through top end 623 and bottom end 626, with the plurality of blades 624 disposed about and/or extending radially outwards from opening 622. Stated differently, main body 621 may be configured such that the plurality of blades 624 are covered along top end 623 by top planar surface 629 and bottom end 626 by bottom planar surface 627. Opening 622 may be sized, shaped, and/or otherwise configured to receive first end 138 of outlet tube 130 and coupling mechanism 111 of upper body 102 similar to opening 128 shown and described above (see FIG. 6).

In the example, the plurality of blades 624 may form at least one slot 638 formed between an adjacent pair of blades 624 between top planar surface 629 and bottom planar surface 627. For example, at least one slot 638 may be positioned along a side of main body 621 between adjacent blades 624, such that agitation mechanism 620 may include a plurality of slots 638 formed below top planar surface 629 and above bottom planar surface 627. In other words, adjacent blades 624 may form a cavity therebetween defining slot 638. Each of the plurality of slots 638 may be sized, shaped, and/or otherwise configured to capture a portion of the pressurized fluid received in enclosure 101 from inlet 108 for release towards lower body 150 as blades 624 rotate, such as in the cyclonic motion to enhance agitation and movement of the agent stored therein.

Agitation mechanism 620 may include a plurality of arms 628 extending relatively downwards from bottom planar surface 627 towards bottom interior surface 154 when agitation mechanism 620 is disposed within enclosure 101. The plurality of arms 628 may include a longitudinal length defined between an upper end 630 and a lower end 632 that is positioned opposite to upper end 630. The plurality of arms 628 may be sized and/or shaped such that at least a lower portion of arms 628 (e.g., lower ends 632) may extend into and/or be disposed inside lower body 150 as an upper portion of arms 628 (e.g., upper ends 630) is disposed inside upper body 102. In some embodiments, arms 628 may include a longitudinal length that terminates proximal to (i.e., relatively above) a surface level of the agent stored in lower body 150. In this instance, arms 628 may not be in contact with and/or abut against the agent. Accordingly, agitation mechanism 620 may be configured to move (e.g. rotate) within enclosure 101 with a lower threshold of force from the pressurized fluid. In the example, the plurality of arms 628 may include a substantially linear configuration that is generally transverse (e.g., perpendicular) to the planar configuration of bottom planar surface 627.

The plurality of arms 628 may be configured to extend relatively downwards from bottom planar surface 627 towards bottom interior surface 154 of the enclosure 101. Lower end 632 of each of the plurality of arms 628 may be separated from one another, and suspended within lower body 150 when agitation mechanism 620 is disposed within enclosure 101. In the example, agitation mechanism 620 may include a pair of arms 628 positioned relative to main body 621 such that arms 628 are disposed along opposite sides of outlet tube 130 when outlet tube 130 is coupled to agitation mechanism 620. The pair of arms 628 may be configured to agitate the agent within enclosure 101 and/or combine the agent with the pressurized fluid by rotating within enclosure 101 to create a vortex. In some embodiments, the pair of arms 628 may not abut against and/or contact the agent. As the mixture of agent and pressurized fluid moves within enclosure 101, outlet tube 130 may be configured to receive the mixture at second end 142 and through channel 140 for delivery towards outlet 104 of enclosure 101 for delivery.

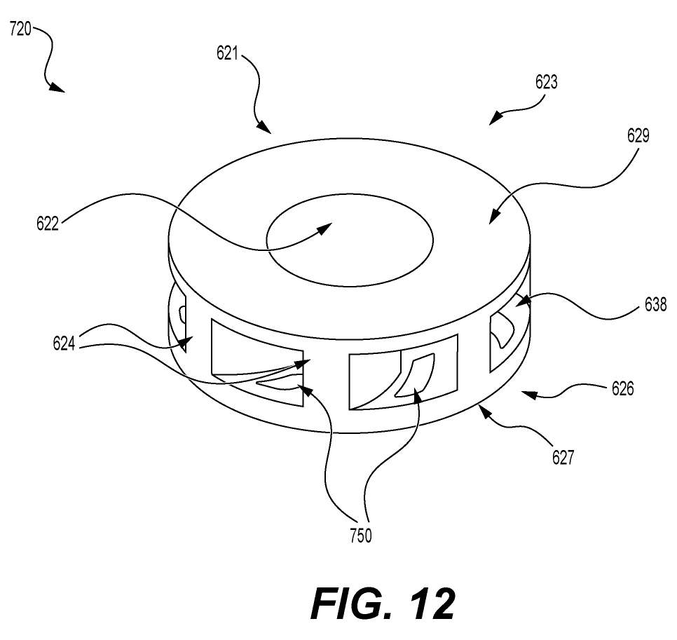

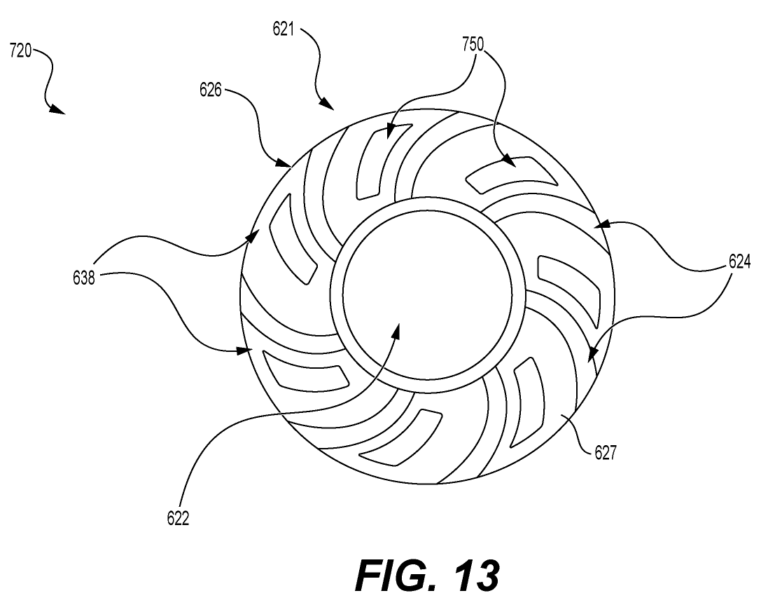

FIGS. 12-13 show another exemplary agitation mechanism 720 of valve assembly 100. It should be appreciated that agitation mechanism 720 may be configured and operable in a substantially similar manner as agitation mechanism 120 shown and described above, such that valve assembly 100 may include agitation mechanism 720 in place of agitation mechanism 120 without departing from a scope of this disclosure. Agitation mechanism 720 may include substantially similar features as agitation mechanisms 120, 620 except for the differences explicitly described herein. Accordingly, like reference numerals are used to describe similar components.

For example, agitation mechanism 720 may include main body 621 having top end 623, bottom end 626 opposite top end 623, the plurality of blades 624 disposed between top end 623 and bottom end 626, and opening 622 extending through the central axis of main body 621 through top planar surface 629 and bottom planar surface 627. Agitation mechanism 720 may be sized, shaped, and/or otherwise configured as a rotatable propeller.

Actuation mechanism 720 may include a plurality of vents 750 formed along bottom end 626, and particularly through bottom planar surface 627. Each of the plurality of vents 750 may be disposed between a set of adjacent blades 624 of the plurality of blades 624. Each of the plurality of vents 750 may be accessible from a corresponding slot 638 defining the cavity formed between adjacent blades 624 on main body 621. In some embodiments, vents 750 may include a curved and/or angled configuration relative to bottom planar surface 627, such that each of the plurality of vents 750 may include a curvature and/or arc relative to the planar configuration of bottom planar surface 627, as best seen in FIG. 13. As described in detail herein, vents 750 may be configured to agitate the pressurized fluid in a direction towards the agent stored in enclosure 101.

Still referring to FIGS. 12-13, the plurality of vents 750 may be sized, shaped, and/or otherwise configured to guide, direct, and/or propel at least a portion of the pressurized fluid received within the corresponding slot 638 between adjacent blades 624 through bottom end 626 and towards lower body 150 as agitation mechanism 720 rotates about outlet tube 130. Vents 750 may be configured to agitate and/or mix the pressurized fluid and agent stored within enclosure 101 by guiding the portion of pressurized fluid through bottom end 626 and towards lower body 150. The plurality of vents 750 may be configured to generate the cyclonic motion of pressurized fluid that is propelled downwards through enclosure 101 towards lower body 150. A size, shape, and/or configuration of the plurality of vents 750 may be configured to generate a turbulent flow of pressurized fluid and agent within enclosure 101 upon movement (e.g., rotation) of agitation mechanism 720.

For example, the pressurized fluid received in enclosure 101 from the fluid source (nots shown) may contact agitation mechanism 720, thereby rotating agitation mechanism 720 about outlet tube 130 within enclosure 101. As agitation mechanism 720 rotates, portions of the pressurized fluid may be received within slots 638 and directed through the plurality of vents 750 positioned between each set of adjacent blades 624 towards bottom interior surface 154 of enclosure 101, thereby agitating the agent stored within enclosure 101 and generating the mixture of agent and pressurized fluid.

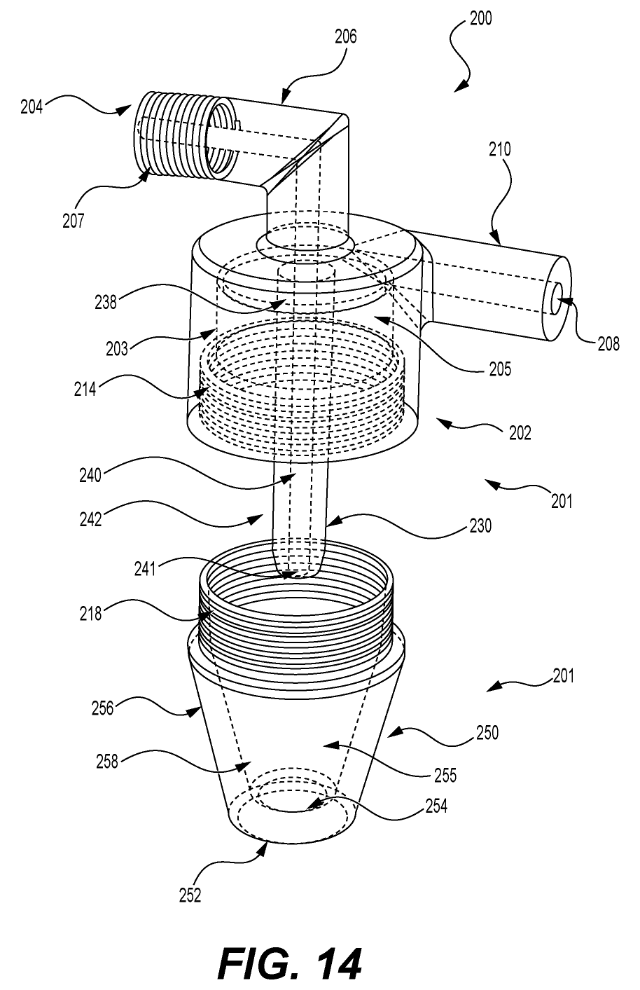

FIG. 14 shows another exemplary valve assembly 200, according to one or more embodiments. Valve assembly 200 may be part of delivery system 10 as discussed with reference to FIG. 1 above, such as disposed in enclosure 14. Valve assembly 200 may be configured and operable similar to valve assembly 100 shown and described above except for the differences explicitly described herein. Valve assembly 200 may include an enclosure 201 for storing the agent and an outlet tube 230 disposed inside enclosure 201. A predetermined amount of agent (e.g., 3 mg to 5 mg) may be stored within enclosure 201. The predetermined amount of agent stored therein may vary depending on a desired application of use, such as a treatment of a subject (e.g., patient) or a target treatment site. Enclosure 201 may include an upper body 202 and a lower body 250 that is removably coupled to upper body 202.

Upper body 202 may include an upper interior surface 203 defining an inner cavity 205, an outlet 204 including a first fastening mechanism 207, and an outlet conduit 206 in fluid communication with inner cavity 205. First fastening mechanism 207 may be disposed around an outer surface of outlet 204, and configured to connect valve assembly 200 to a catheter (not shown). Upper body 202 may further include an inlet 208, an inlet conduit 210 in fluid communication with inner cavity 205, and a second fastening mechanism 214, with each fastening mechanism 207, 214 being configured for removably coupling one or more components of valve assembly 200 to upper body 202.

Inlet 208 may be configured to receive pressurized fluid from the fluid source of delivery device 10 (see FIG. 1) for distribution into enclosure 201. Inlet 208 may be sized, shaped, and/or otherwise configured to direct the pressurized fluid towards upper interior surface 203 at a predefined angle. In other words, inlet 208 may be arranged relative to upper interior surface 203 such that inlet 208 may be configured to guide the pressurized fluid through inlet conduit 210 and along upper interior surface 203 upon receipt within inner cavity 205. Upper interior surface 203 of upper body 202 may include a cylindrical cross-sectional profile that varies relative to upper body 102, such that upper body 202 may be sized and/or shaped to guide the pressurized fluid entering into enclosure 201 in a clockwise or counterclockwise rotative direction towards lower body 250 at a stream angle of flow that varies relative to valve assembly 100 shown and described above.

Still referring to FIG. 14, lower body 250 of enclosure 201 may include a third fastening mechanism 218, a bottom exterior surface 252, a bottom interior surface 254, a side exterior surface 256, and a side interior surface 258. Bottom interior surface 254 and side interior surface 258 of lower body 250 may collectively define an inner cavity 255 of lower body 250. Lower body 250, and particularly inner cavity 255, may include a cyclonic configuration and/or tapered cross-sectional profile that is at least partially defined by side interior surface 258. In other words, side interior surface 254 may include a tapered cross-sectional profile such that lower body 250 may be sized and/or shaped to guide the pressurized fluid received from upper body 202 in a clockwise or counterclockwise rotative direction.

Upper body 202 may be rotatably coupled to lower body 250 via an engagement between second fastening mechanism 214 and third fastening mechanism 218. In the embodiment, fastening mechanisms 207, 214, and 218 may include one or more threaded regions that are sized, shaped, and/or otherwise configured to threadably mate and/or couple with one another and/or other components of delivery device 10. Although fastening mechanisms 207, 214, and 218 may be shown and described herein as threaded regions, it should be appreciated that various other suitable fastening mechanisms may be incorporated into valve assembly 200 without departing from a scope of this disclosure. For example, the fastening mechanisms may include, but are not limited to, one or more clamps, clips, tabs, protrusions, arms, etc.

Still referring to FIG. 14, outlet tube 230 may integrally mounted to upper body 202 such that outlet tube 230 forms a unitary structure with upper body 202. Outlet tube 230 may include a longitudinal length defined between a first end 238 and a second end 242 that is positioned along the longitudinal length of outlet tube 230 opposite to first end 238. Outlet tube 230 may include a channel 240 having a longitudinal length defined between first end 238 and a second opening 241 at second end 242.

In other words, the longitudinal length of channel 240 may correspond to the longitudinal length of outlet tube 230 defined between first end 238 and second end 242. Outlet tube 230 may be in fluid communication with outlet conduit 206 via channel 240. As described further herein, channel 240 may be configured to receive a mixture of the pressurized fluid received in enclosure 201 via inlet 208 and the agent stored in enclosure 201 along bottom interior surface 254 at second opening 241, and direct the mixture through channel 240 for delivery towards outlet conduit 206. In other words, channel 240 may be configured to move the mixture through second opening 241 and towards outlet conduit 206, thereby allowing for fluid communication of outlet tube 230 and outlet 204 for delivering the mixture out of enclosure 201.

As described above, inlet 208 may be configured to direct the pressurized fluid into upper body 202, and particularly within the cyclonic configuration of inner cavity 205. As such, a motion of movement of the pressurized fluid within upper body 202 may conform to the cyclonic configuration of inner cavity 205 as defined by upper interior surface 203. In the embodiment, a configuration of inner cavity 205 and/or inner cavity 255 may provide a turbulent flow of pressurized fluid directed towards the agent stored along bottom interior surface 254. The cyclonic motion of pressurized fluid may include, but is not limited to, a circular movement, a twisting movement, a swirling movement, a rotative movement, a funnel-like movement, and/or a spiral movement within inner cavity 205 and/or inner cavity 255. In response to agitating the agent within enclosure 201, outlet tube 230 may be configured to receive the mixture of pressurized fluid and agent via second opening 241 at second end 242, and direct the mixture upwards through channel 240 for delivery into outlet 204 that is fluidly coupled channel 240 via outlet conduit 206.

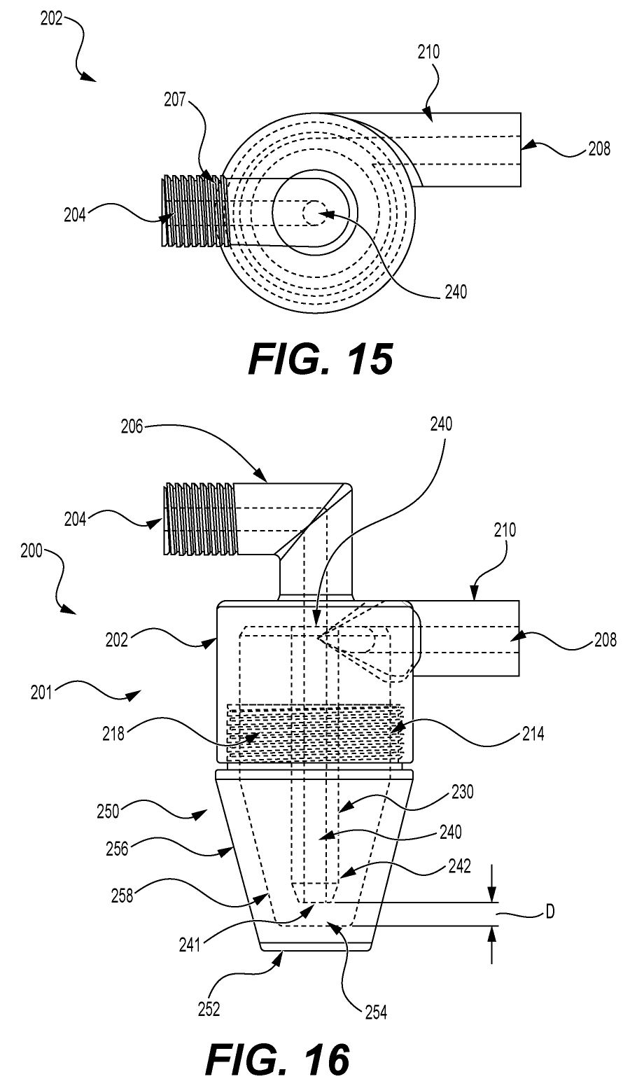

As best seen in FIGS. 15-16, inlet 208 may be positioned relative to upper body 202 such that the pressurized fluid received into enclosure 201 via inlet 208 is directed against upper interior surface 203. In other words, upon entry of the pressurized fluid into enclosure 201 through inlet 208, inner cavity 205 may be configured to capture the pressurized fluid along upper interior surface 203 facing inlet conduit 210, thereby causing the pressurized fluid to move in a cyclonic motion. By generating the cyclonic motion of pressurized fluid, upper body 202 and lower body 250 may be configured to agitate the agent stored within enclosure 201.

In some embodiments, the cyclonic motion of pressurized fluid generated by upper body 202 and lower body 250 may combine the pressurized fluid and agent, such that valve assembly 200 may be configured to create a mixture of pressurized fluid and agent. Outlet tube 230 may be configured to receive the mixture into second opening 241 at second end 242. In the example, lower body 250 and outlet tube 230 may be sized, shaped, and/or otherwise configured to form gap D between second end 242 and bottom interior surface 254 when upper body 202 is coupled to lower body 250. For example, outlet tube 230 may have a longitudinal length that positions second end 242 adjacent to bottom interior surface 254, with gap D defining an offset distance disposed therebetween.

Still referring to FIG. 16, valve assembly 200 may include gap D disposed between second end 242 of outlet tube 230 and bottom interior surface 254 of lower body 250. Outlet tube 230 may be configured to direct the mixture of pressurized fluid and agent through channel 240 and towards outlet 204 due to second opening 241 at second end 242 being separated from bottom interior surface 254 by gap D. It should be appreciated gap D may include various suitable dimensions without departing from a scope of this disclosure, as described in detail above.

Although valve assemblies 100, 200 and agitation mechanisms 120, 320, 420, 520, 620, 720 are shown and described herein as being implemented in delivery system 10, and particularly in a handheld device, it should be appreciated that they may be configured and operable in various other suitable configurations. For example, valve assemblies 100, 200 may be directly coupled to a delivery device (e.g., an endoscope, a catheter, etc.) that is positioned adjacent to a target treatment site within a subject (e.g., a patient).

While principles of this disclosure are described herein with reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Accordingly, the invention is not to be considered as limited by the foregoing description. The features described herein may be used alone or in combination and are not mutually exclusive.

What is claimed is:

1. A device for delivering an agent, comprising:
a valve assembly including an enclosure for storing the agent, an agitation mechanism rotatably disposed inside the enclosure, and a tube disposed inside the enclosure and extending through the agitation mechanism;
wherein the tube includes a channel having a longitudinal length defined between a first opening at a first end of the tube and a second opening at a second end of the tube;
wherein the agitation mechanism is configured to direct a fluid received in the enclosure towards the agent stored therein in response to rotating about the tube, thereby agitating the agent within the enclosure;
wherein the tube is configured to receive a mixture of the fluid and the agent at the second opening and direct the mixture through the channel for delivery out of the enclosure via the first opening.

2. The device of claim 1, wherein the agitation mechanism includes:
a propeller including a main body; and
a plurality of blades extending radially outwards from the main body.

3. The device of claim 2, wherein the main body includes:
a top end including a planar surface covering the plurality of blades and an opening extending through the planar surface, the opening being configured to surround the first end of the tube; and
a bottom end that is open to expose the plurality of blades, the bottom end including a seat disposed adjacent to the bottom end along the plurality of blades.

4. The device of claim 2, wherein the main body includes:
a top end that is open to expose the plurality of blades, the top end including an opening configured to surround the first end of the tube; and
a bottom end including a planar surface covering the plurality of blades, wherein the opening extends through the planar surface;
wherein the agitation mechanism includes a base and a plurality of arms extending between the main body to the base, the plurality of arms being configured to agitate the agent within the enclosure in response to the agitation mechanism rotating about the tube.

5. The device of claim 4, wherein the base is offset from the second end of the tube to form a gap, wherein the base is configured to direct the mixture into the channel for delivery from the tube via the gap.

6. The device of claim 4, wherein the base includes:
an agitator extending outwardly from the base towards the second end of the tube, the agitator being configured agitate the agent within the enclosure in response to the agitation mechanism rotating about the tube.

7. The device of claim 4, wherein each of the plurality of arms includes a helical configuration extending between the main body and the base.

8. The device of claim 2, wherein the main body includes:
a top end including a top planar surface covering the plurality of blades and an opening extending through the top planar surface, the opening being configured to surround the first end of the tube;
a bottom end including a bottom planar surface covering the plurality of blades such that the plurality of blades is disposed between the top planar surface and the bottom planar surface, wherein the opening extends through the bottom planar surface.

9. The device of claim 8, wherein the main body includes at least one slot formed between a pair of adjacent blades of the plurality of blades, wherein the at least one slot is configured to direct the fluid received within the main body outwards from between the top planar surface and the bottom planar surface towards the agent.

10. The device of claim 9, wherein the main body includes at least one vent formed within the at least one slot between each of the pairs of adjacent blades, wherein the at least one vent is configured to direct the fluid received within the at least one slot downwards through the bottom planar surface towards the agent.

11. The device of claim 1, wherein the tube includes a platform between the first end and the second end, and the platform is configured to receive the agitation mechanism on the tube.

12. The device of claim 1, wherein the enclosure includes a first fastening mechanism, and the tube includes a second fastening mechanism at the first end that is configured to removably couple the tube to the enclosure, thereby suspending the second end of the tube inside the enclosure.

13. The device of claim 12, wherein the tube extends towards a bottom surface of the enclosure to create a gap between the second end of the tube and the bottom surface, and the tube is configured to receive the mixture within the channel via the gap.

14. The device of claim 1, wherein the enclosure includes an upper body and a lower body that are removably coupled to one another to form a cavity, and the tube and the agitation mechanism are disposed inside the cavity.

15. The device of claim 1, further comprising a connector assembly removably coupled to the valve assembly, wherein the connector assembly includes a port configured to receive an instrument to clear an outlet of the device of residual agent.

16. A device for delivering an agent, comprising:
a valve assembly including an upper body, a lower body removably coupled to the upper body to define an enclosure, and a tube disposed inside the enclosure and extending between the upper body and the lower body;
wherein the tube includes a first end mounted to the upper body and a second end opposite to the first end that is suspended inside the lower body to form a gap;
wherein the upper body is configured to guide a pressurized fluid received in the valve assembly towards the lower body in a cyclonic motion, and the lower body is configured to guide the pressurized fluid received from the upper body in the cyclonic motion towards the agent, thereby agitating the agent with the pressurized fluid;
wherein the tube is configured to receive a mixture of the pressurized fluid and the agent at the second end via the gap for delivery from the lower body to the upper body for exiting the enclosure.

17. The device of claim 16, wherein the gap includes an offset distance formed between a bottom surface of the lower body and the second end of the tube.

18. The device of claim 16, wherein the upper body includes an interior surface that is configured to direct the pressurized fluid received within the upper body downwards towards the lower body in the cyclonic motion.

19. A device for delivering an agent, comprising:
a valve assembly including an enclosure, a tube disposed inside the enclosure, and an agitation mechanism rotatably mounted to the tube, the enclosure is defined by an upper body that is configured to receive a pressurized fluid and a lower body that is configured to store the agent;
wherein the agitation mechanism is configured to rotate about the tube in response to the upper body receiving the pressurized fluid to propel the pressurized fluid downwards towards the agent stored in the lower body;
wherein the tube includes a channel that is in fluid communication with the lower body, such that the tube is configured to receive a mixture of the pressurized fluid and the agent within the channel for delivery out of the enclosure.

20. The device of claim 19, wherein the agitation mechanism includes a propeller having a main body and a plurality of blades extending radially outwards from the main body, each of the plurality of blades including an interior surface that is configured to capture the pressurized fluid, thereby causing the main body to rotate around the tube.
